(19) United States
(12) Patent Application Publication
(10) Pub. No.: US 2011/0206751 A1
(43) Pub. Date: Aug. 25, 2011

(54) STIMULATABLE ASYMMETRICAL POLYMERSOME

(75) Inventors: Min-Hui Li, Antony (FR); Elyes Mabrouk, Paris (FR); Damien Cuvelier, Paris (FR); Pierre Nassoy, Suresnes (FR); Françoise Brochard-Wyart, Saint-cloud (FR)

(73) Assignees: INSTITUT CURIE, Paris Cedex 05 (FR); CENTRE NATIONAL DE LA RECHERCHE SCIENTIFIQUE, Paris Cedex 16 (FR); UNIVERSITE PIERRE ET MARIE CURIE, Paris Cedex 05 (FR)

(21) Appl. No.: 13/119,340

(22) PCT Filed: Sep. 15, 2009
(86) PCT No.: PCT/IB2009/054033

§ 371 Date: May 10, 2011

(30) Foreign Application Priority Data
Sep. 16, 2008 (FR) 0856227
Oct. 9, 2008 (FR) 0856849

(51) Int. Cl. A61K 8/11; A61K 8/90; A61K 9/00; A61P 17/00; A61Q 19/00; B32B 27/08; C08L 53/00; B01J 13/18; A61K 9/50

(52) U.S. Cl. 424/401; 424/400; 428/519; 428/402; 525/89; 264/4.7; 264/4.32

(57) ABSTRACT

The invention relates to a polymer vesicle with an asymmetrical unilamellar membrane, said membrane being constituted by two distinct layers, a layer (A) and a layer (B), said layers comprising respective amphiphilic block copolymers with layer (A) comprising, in contrast to said layer (B), an effective quantity of amphiphilic block copolymer comprising at least one stimulatable hydrophobic polymer block, said block polymers provided by said copolymers being capable of adopting a new steric configuration in response to an exogenous stimulus, conditioning the rupture of said unilamellar membrane. The invention also provides a method of producing said vesicle, a method of encapsulating an active ingredient in said vesicles, and a method of controlled release of the active ingredient.

The present invention relates to vesicles, in particular polymersomes, intended to encapsulate, to transport, and to provide for the controlled release of one or more hydrophilic active ingredients.

The term “polymersome” means vesicles defined by a membrane formed from amphiphilic synthetic polymers. Below, the expressions “polymersome” or “vesicle with a unilamellar membrane” are used interchangeably to denote the same entity.

From a structural standpoint, polymersomes resemble liposomes, the only difference being that liposomes are produced from lipids. Polymersomes possess most of the properties of liposomes, but in addition they have greater stability and lower permeability.

In general, polymersomes have a unilamellar membrane. This unilamellar membrane is termed “symmetrical” when the two superposed layers that form it are constituted by identical copolymers. In contrast, an “asymmetrical” unilamellar membrane has two superposed layers that are distinguished from each other by the specific natures of the copolymers that constitute them. This difference between said two types of copolymer may reside in the nature of the hydrophobic block and/or in the nature of the hydrophilic block forming the copolymers.

The development of polymersomes is of especial interest in the fields of encapsulation, transport, and controlled release. Further, the presence on the periphery of such vesicles of an outer layer of biocompatible hydrophilic blocks, which may advantageously be of the polyethylene glycol type, can render these polymersomes furtive as regards the immune system. This characteristic, which is also associated with a certain form of harmlessness, makes them ideal transporters or vectors for active ingredients, for example chemical, cosmetic, dermatological, and/or pharmaceutical ingredients.

The present invention seeks specifically to propose polymersomes of a novel type that allow effective control of release of the active ingredient they contain.

Illustrative examples of controlled release polymersomes that have already been proposed and that may be mentioned in particular are symmetrical unilamellar polymersomes comprising a double layer of block copolymer(s), which are amphiphilic and sensitive to hydrolysis, for example polyethylene glycol-polylactic acid or polyethylene glycol-polycaprolactone block copolymers, and which may optionally be mixed with polyethylene glycol-polybutadiene block copolymers described in document US 2005/0003016. The polymersomes therein are described as being for use in containing active ingredients such as drugs that are released in a controlled manner following hydrolysis of the copolymers in the membrane. This release is conditioned by an environmental stimulus and involves a stage whereby the unilamellar membrane is perforated to reach a stage of total disintegration of the vesicle, which stage nevertheless extends over a relatively long time period that is counted in hours or even day(s) and is thus not always compatible with the desired applications.

Another type of symmetrical unilamellar polymersome having a bilayer that comprises amphiphilic, heat-sensitive block copolymers is described in document WO-2007/075502. Those polymersomes are also capable of containing active principles and of transporting them to target organs. The copolymers are of the polyethyleneglycol-b-poly(N-alkylacrylamide) or polyethyleneglycol-b-poly(N-alkylaminoacrylate) type. Beyond a certain critical solution temperature, the heat-sensitive blocks have hydrophobic properties that result in them self-assembling to form a polymersome. In contrast, below that temperature, the heat-sensitive blocks regain hydrophilic properties, resulting in dissociation of said polymersome. The critical solution temperature of heat-sensitive block copolymers of the poly(N-isopropyl acrylamide) type is approximately 32° C. However, using that type of controlled release system poses a certain number of problems, in particular in vivo, namely that the temperature of the polymersomes must be taken to below 32° C., i.e. well below body temperature, which is 37.2° C., by means of a cooling patch so that it is possible for them to release the active principle they enclose.

Thus, there remains a need for polymersomes that, in response to an external stimulus that is readily applicable in a topical manner and/or in vivo, are capable of giving rise to tailored, controlled, virtually instantaneous, and complete release of their contents.

The inventors have now discovered that it is possible to provide polymersomes or vesicles with a synthetic unilamellar membrane that satisfy these criteria by specific selection of the copolymers.

First exemplary embodiments of the invention provide a vesicle with an asymmetrical unilamellar membrane, said membrane being constituted by two distinct layers, a layer (A) and a layer (B), said layers being superposed and each comprising at least one respective amphiphilic block copolymer, the vesicle being characterized in that layer (A) comprises an effective quantity of amphiphilic block copolymer(s) comprising at least one block that is capable of being stimulated by an exogenous stimulus, said stimulatable blocks provided by said copolymers being capable of adopting, in response to said exogenous stimulus, a novel steric configuration conditioning rupture of said unilamellar membrane, and in that said layer (B) does not have an effective quantity of amphiphilic block copolymer(s) comprising at least one block that is capable of being stimulated by said exogenous stimulus.

More precisely, layers (A) and (B) formed by amphiphilic block copolymers constituting said unilamellar membrane are superposed head to head, i.e. such that the hydrophobic blocks in said copolymers constitute the inner face of said membrane and the hydrophilic blocks in said copolymers constitute the outer face of said membrane.

In preferred exemplary embodiments, the vesicle of the invention further includes at least one active ingredient.

Second exemplary embodiments of the invention provide a composition, in particular chemical, cosmetic, dermatological, and/or pharmaceutical composition, comprising at least one vesicle in accordance with the invention, optionally in a hydrophilic or physiologically acceptable medium.

The term “physiologically acceptable medium” means a non-toxic medium that is compatible with ex vivo application, for example onto keratinous material, or in vivo, for example by ingestion by a living organism, in particular human or animal.

Third exemplary embodiments of the invention provide a method of manufacturing vesicles with an asymmetrical unilamellar membrane in accordance with the invention, the method being characterized in that it comprises at least the steps consisting in:

a) dispersing an aqueous phase in an oily phase in the presence of at least one amphiphilic block copolymer (X) under conditions propitious to the formation of a layer of said amphiphilic block copolymer (X) around aqueous droplets;

b) bringing said coated droplets obtained at the end of the preceding step into contact with at least one amphiphilic block copolymer (Y), which is different from the amphiphilic block copolymer (X), under conditions propitious to the formation of a layer comprising said amphiphilic block copolymer (Y) superposed on the layer formed in step a); and

c) recovering said vesicles with an asymmetrical unilamellar membrane in an aqueous phase; characterized in that only one of steps a) and b) uses, as the amphiphilic block copolymer, an effective quantity of at least one amphiphilic block copolymer comprising at least one block that is capable of being stimulated by an exogenous stimulus.

In an implementation, the layer of step b) is formed at the interface of an oily phase and an aqueous phase.

For obvious reasons, the amphiphilic block copolymer comprising at least one block that is capable of being stimulated by an exogenous stimulus is used in the step under consideration in a quantity sufficient to be able to provoke rupture of the membrane of the polymersome which it forms, in response to exposure of the polymersome to the exogenous stimulus to which said copolymer is receptive.

In contrast, in the other step, such a copolymer is present in a quantity that is not effective, or even is completely absent.

Thus, in a variation, only one of its two steps employs, as the amphiphilic block copolymer, at least one amphiphilic block copolymer comprising at least one stimulatable block.

In an implementation of the production method, the layer of step b) is formed at the interface of an oily phase and an aqueous phase.

Fourth exemplary embodiments of the invention provide a method of encapsulating at least one hydrophilic active ingredient, in particular a cosmetic, dermatological or pharmaceutical molecule, a polymer and/or a chemical reagent, in vesicles in accordance with the invention, the method being characterized in that it comprises at least the steps consisting in:

a) dispersing an aqueous phase comprising at least one active ingredient in an oily phase in the presence of at least one amphiphilic block copolymer (X) under conditions that are propitious to the formation of a layer of said amphiphilic block copolymer (X) around aqueous droplets containing said active ingredient or active ingredients;

b) bringing said coated droplets obtained from the preceding step into the presence of at least one amphiphilic block copolymer (Y), which is different from the amphiphilic block copolymer (X), under conditions propitious to the formation of a layer comprising said amphiphilic block copolymer (Y) superposed on the layer formed in step a); and

c) recovering said vesicles with an asymmetrical unilamellar membrane containing said active ingredient or active ingredients in an aqueous phase; the two steps a) and b) being as defined above.

Thus, only one of the two steps a) or b) advantageously employs, as the amphiphilic block copolymer, at least one amphiphilic block copolymer comprising at least one stimulatable block.

Fifth exemplary embodiments of the invention provide a method of the controlled release of at least one active ingredient contained in a vesicle with an asymmetrical unilamellar membrane in accordance with the invention, the method consisting in exposing said vesicle to an exogenous stimulus capable of inducing a modification to the steric configuration of the amphiphilic block copolymers comprising at least one block that is capable of being stimulated by said exogenous stimulus, under conditions sufficient to provoke the rupture of said unilamellar membrane of said vesicle.

Final exemplary embodiments of the invention provide the use of a vesicle in accordance with the invention for the purposes of encapsulating, transporting, vectorizing and/or releasing at least one active ingredient ex vivo, in vivo, or in vitro.

### Polymersome

A vesicle with a unilamellar polymeric membrane, or polymersome, in accordance with the invention may have a mean diameter in the range 100 μm [micrometer] to 20 nm [nanometer], or even in the range 75 μm to 50 nm and more particularly in the range 50 μm to 100 nm.

A vesicle of the invention comprises a unilamellar membrane constituting the shell of said vesicle and a core forming a liquid phase, in particular an aqueous liquid phase, advantageously formed entirely or partially of water. This is termed a “core/shell” type structure.

Regarding the core, the liquid phase may be solely constituted by water or it may comprise an aqueous solution, i.e. a mixture of water with one or more hydrosoluble solvent(s).

The term “hydrosoluble solvent” as used in the present invention means a compound that is liquid at ambient temperature and miscible with water (miscibility in water greater than 50% by weight at 25° C. and at atmospheric pressure).

Examples of hydrosoluble solvents that may be used as the liquid phase forming the core of the vesicle of the invention and that may be mentioned are lower alcohols containing 1 to 5 carbon atoms such as ethanol or isopropanol, glycols containing 2 to 8 carbon atoms such as ethylene glycol, propylene glycol, 1,3-butylene glycol or dipropylene glycol, C₃ and C₄ ketones, and C₂-C₄ aldehydes.

The unilamellar membrane is constituted by two layers: a first layer termed the “inner layer” that is in direct contact with the core of said vesicle, and a second layer termed the “outer layer” that is superposed on and thus contiguous with said first layer and that is in direct contact with the medium in which said vesicle is found in order to form a structure of the “outer medium/outer layer/inner layer/core” type.

The hydrophilic blocks are oriented such that they come into contact with the media for which they have the most affinity. Thus, in the inner layer they are oriented towards the aqueous liquid phase core of said vesicle and in the outer layer they are oriented towards the external medium in which said vesicle is immersed, while the hydrophobic blocks are positioned inside the shell of said vesicle so as to form a structure of the “outer medium/hydrophilic blocks/hydrophobic blocks/hydrophobic blocks/hydrophilic blocks/core” type.

As discussed above, the unilamellar membrane of the vesicle of the invention is also symmetrical, i.e. it is constituted by a layer (A) and a superposed layer (B) that differ from each other. In the text, the expressions “vesicle with an asymmetrical unilamellar membrane” and “asymmetrical polymersome” are used interchangeably.

### Layer (A)

In the context of the present invention the layer (A) is defined as the layer containing the amphiphilic block copolymer or copolymers comprising at least one polymer block that is capable of adopting a new steric configuration in response to an exogenous stimulus. Such a block is termed a “stimulatable block”.

For simplification purposes, such an amphiphilic block copolymer comprising at least one stimulatable block is also termed a “stimulatable amphiphilic block copolymer” or “stimulatable block copolymer” or “stimulatable amphiphilic copolymer” or even “stimulatable copolymer”, all of these expressions being equivalent.

An “effective quantity of stimulatable copolymer” means a sufficient quantity of said copolymer or copolymers to allow rupture of the membrane of said vesicle consecutive to a change in the steric configuration of said stimulatable copolymer in response to exposure of said copolymer to an exogenous stimulus.

As can be seen from the above, said stimulatable block contains at least one motif termed a “stimulatable motif”, which is sensitive to an exogenous stimulus.

In a preferred variation of the invention, a stimulatable copolymer comprises at least one stimulatable block, advantageously with a liquid crystal nature, termed a “liquid crystal block”.

The stimulatable motifs with a liquid crystal nature are capable of modifying their structural organization (orientation, mesomorphous phase) in response to an external stimulus, in particular light radiation, a magnetic field, an electrical field, or temperature.

More particularly, it is a photo-stimulatable liquid crystal block composed of at least one motif containing the azobenzene group.

The term “photo-stimulatable” means capable of undergoing modifications in response to exposure to light radiation.

Thus, in response to exposure to light radiation, a motif containing at least one azobenzene group is capable of undergoing a change in configuration from a trans configuration to a cis configuration. This configurational change in a motif has the result of inducing a new steric configuration (shape) in said stimulatable block. The overall effect of these structural modifications is to provoke, in the unilamellar architecture formed in particular by the corresponding copolymers, an increase in the surface area of just one of the two layers and thus a spontaneous change in the curvature of the membrane, which proves to be effective in inducing rapid bursting of the vesicle of the invention.

In preferred exemplary embodiments, the stimulatable copolymer is selected from copolymers comprising at least one hydrophilic block and at least one liquid crystal hydrophobic block. In these particularly advantageous exemplary embodiments, the liquid crystal hydrophobic block is constituted by a photo-stimulatable liquid crystal hydrophobic block comprising at least one azobenzene group.

Particular illustrative, non-limiting examples of this type of block that may be mentioned are the polymeric blocks poly(4-butyloxy-2′-(4-(methacryloyloxy)butyloxy)-4′-(4-butyloxybenzoyloxy)azobenzene), poly(4-butyl-2′-(4-(methacryloyloxy)butyloxy)-4′-(4-butyloxy-benzoyloxy)azobenzene, poly(4-butyloxy-2′-(4-(acryloyloxy)butyloxy)-4′-(4-butyloxy-benzoyloxy)azobenzene, poly(4-butyl-2′-(4-(acryloyloxy)butyloxy)-4′-(4-butyloxy-benzoyloxy)azobenzene, poly(4-alkyloxy-2′-(methacryloyloxyalkyloxy)-4′-(4-alkyloxy-benzoyloxy)azobenzene, poly(4-alkyloxy-2′-(acryloyloxyalkyloxy)-4′-(4-alkyloxy-benzoyloxy)azobenzene, poly(4-alkyl-2′-(methacryloyloxyalkyloxy)-4′-(4-alkyloxy-benzoyloxy)azobenzene, poly(4-alkyl-2′-(acryloyloxyalkyloxy)-4′-(4-alkyloxy-benzoyloxy)azobenzene, poly(4-alkyl-2′-(methacryloyloxyalkyloxy)-4′-(4-alkyl-benzoyloxy)azobenzene, poly(4-alkyl-2′-(acryloyloxyalkyloxy)-4′-(4-alkyl-benzoyloxy)azobenzene, poly(4-alkyloxy-2′-(methacryloyloxyalkyloxy)-4′-(4-alkyl-benzoyloxy)azobenzene, poly(4-alkyloxy-2′-(acryloyloxyalkyloxy)-4′-(4-alkyl-benzoyloxy)azobenzene, poly(6-[4-(4-methoxyphenylazo)phenoxy]hexylmethacrylate, and poly(6-[4-(4-methoxyphenylazo)phenoxy]hexylacroylate.

In this variation, in addition to the photo-stimulatable block or blocks, the copolymer under consideration in layer (A) may comprise at least one other non-stimulatable liquid crystal polymer block.

Particular illustrative, non-limiting examples of this type of non-photo-stimulatable liquid crystal polymer block that may be mentioned are the polymer blocks poly((4″-methacryloxybutyl)2,5-di(4′-butyloxybenzoyloxy)benzoate), poly((6″-methacryloxyhexyl)2,5-di(4′-butyloxybenzoyloxy)benzoate), poly((methacryloxyalkyl)2,5-di(4′-alkyloxybenzoyloxy)benzoate), poly((acryloxyalkyl)2,5-di(4′-alkyloxybenzoyloxy)benzoate), poly(4″-acryloyloxybutyl), and 2,5-di(4′-pentylcyclohexylcarboxyloxy)benzoate.

In this variation, advantage is taken of the fact that the organization of these non-photo-stimulatable liquid crystal polymer blocks changes at the same time and in the same manner as the photo-stimulatable liquid crystal blocks when these react in response to exposure to light radiation. The steric configuration (shape) of all of the liquid crystal polymer blocks changes thereby, inducing a spontaneous change in the curvature of the membrane so as to cause said membrane to rupture and ultra-rapid bursting of said vesicle.

To render the photo-stimulatable response particularly effective, layer (A) may comprise 10% to 100% by weight of photo-stimulatable liquid crystal polymer(s).

The copolymer under consideration in layer (A) may also comprise at least one other block that is not stimulatable by an external stimulus and in particular one selected from those proposed below for layer (B). Whatever the situation, the chemical nature of the different types of polymeric blocks forming the stimulatable copolymer is adjusted to provide them with the amphiphilic nature required for the invention.

In accordance with an advantageous variation, the stimulatable amphiphilic copolymer comprises at least one hydrophilic polymeric block selected from polyethylene glycol, polyacrylic acid, polymethacrylic acid, poly(N-alkylacrylamide), poly(N-alkylaminoacrylate), poly(acrylic acid-co-oligo(ethylene glycol) acrylate), poly(methacrylic acid-co-oligo(ethylene glycol)methacrylate), poly(oligo(ethylene glycol)methacrylate), poly(oligo(ethylene glycol)acrylate), and poly(2-(2′-methoxyethoxy)ethylmethacrylate-co-oligo(ethylene glycol)methacrylate).

Examples of stimulatable amphiphilic copolymers suitable for the invention that may in particular be mentioned are the block copolymers polyethylene glycol-b-poly(4-butyloxy-2′-(4-methacryloxyloxy)butyloxy)-4′-(4-butyloxy-benzoyloxy)azobenzene.

The number average molecular mass of the stimulatable copolymer or copolymers may be in the range 2000 to 30000, advantageously in the range 3000 to 10000.

It should be understood that layer (A) comprising the stimulatable copolymer or copolymers may also additionally contain at least one other “non-stimulatable” copolymer, selected in particular from those described below for layer (B), provided that the presence of said non-stimulatable copolymer or copolymers does not constitute an obstacle to manifestation of the steric change of said stimulatable copolymer or copolymers.

Layer (A) may include more than 10% by weight, in particular in the range 20% to 95% by weight, in particular in the range 30% to 70% by weight, or even more than 40% by weight of stimulatable amphiphilic block copolymers relative to the total weight of the copolymers in said asymmetrical unilamellar membrane.

### Layer (B)

In contrast to layer (A), layer (B) does not contain an effective quantity of stimulatable copolymer(s) capable of provoking rupture of the membrane of the vesicle under the effect of an exogenous stimulus that can stimulate layer (A).

In other words, layer (B) is essentially formed from copolymers that are inert to the exogenous stimulus to which the copolymer or copolymers forming layer (A) is or are active.

In particular, relative to the total weight of copolymers in said unilamellar membrane, layer (B) may comprise less than 50% by weight, in particular less than 30% by weight of stimulatable amphiphilic block copolymer(s), or the stimulatable amphiphilic block copolymer may even be absent.

Consequently, layer (B) comprises at least one amphiphilic block copolymer that is insensitive to the exogenous stimulus selected to stimulate the layer (A).

For the purposes of simplification, the amphiphilic block copolymer that is insensitive to an exogenous stimulus selected to stimulate the layer (A) is also termed below as a “non-stimulatable amphiphilic block copolymer”, or a “non-stimulatable block copolymer”, or a non-stimulatable amphiphilic copolymer”, or even a “non-stimulatable copolymer”, with all of these expressions being equivalent.

In particular and without limitation to the composition, such a copolymer may be formed from polymeric blocks selected from the following hydrophilic and hydrophobic polymers:

homopolymers and random copolymers of the polyoxyalkylene type such as polyoxypropylene, polyoxybutylene, polyoxyethylene (polyethylene glycol equivalent), polyoxyethylene/polyoxypropylene or polyoxyethylene/polyoxybutylene;

polyacrylic derivatives deriving from homopolymerization or copolymerization of monomers selected from acrylic and methacrylic acids, alkyl acrylates and methacrylates such as methyl, propyl, n-butyl, tert-butyl, hydroxypropyl or hydroxyethyl acrylates and methacrylates, oligo(ethylene glycol) acrylates et methacrylates, N-alkyl-acrylamides or -methacrylamides such as N-ethylacrylamide, N-isopropylacrylamide, N,N-dialkylacrylamides or methacrylamide N,N-dialkylacrylamides;

polyethers;

polyesters such as polyglycolic acid;

cellulose derivatives such as hydroxyalkylcelluloses, for example hydroxyethylcellulose or methylcellulose;

polysaccharides;

polyvinyl alcohol;

polyvinylpyrrolidone;

polystyrenesulfonate;

polysulfoxides;

alkylene homo- and copolymers such as butylene-propylene, ethylene-propylene, ethylene-butylene, poly(ethylethylene) or poly(butadiene);

polyvinylalkylethers such as polyvinylmethylether;

poly(styrene);

poly(lactic acid);

poly(caprolactone);

polyurethanes;

polyamides; and

polysiloxane derivatives such as polydimethylsiloxane.

The chemical natures of the various types of polymeric blocks forming the non-stimulatable copolymer are adjusted to endow it with the required amphiphilic nature of the invention. This adjustment falls within the competence of the skilled person.

A non-stimulatable copolymer may in particular comprise, as the hydrophilic block, at least one block selected from polyethylene glycol, polyacrylic acid, polymethacrylic acid, polyhydroxyethyl acrylate, oligo(ethylene glycol)acrylate or methacrylate homopolymer, or their copolymers with acrylic or methacrylic acid.

It may also include, as the hydrophobic block, at least one block selected from polystyrene, polyethylethylene, polybutadiene, poly L-lactic acid, polycaprolactone, and mixtures thereof.

Particular illustrative and non limiting examples of non-stimulatable copolymers for use in the invention that may be mentioned are polyethylene glycol-polybutadiene block copolymers, polyacrylic acid-polystyrene block copolymers, polyethylene glycol-polystyrene block copolymers, polyethylene glycol-polyethylethylene block copolymers, polyethylene glycol-polybutadiene block copolymers, polyethylene glycol-poly-L-lactic acid block copolymers, and polyethylene glycol-polycaprolactone block copolymers.

The number average molecular weight of the non-stimulatable copolymer or copolymers may be in the range 2000 to 30000, advantageously in the range 3000 to 10000.

It should be understood that layer (B) comprising the non-stimulatable copolymer or copolymers may also further contain at least one copolymer containing a stimulatable polymeric block selected in particular from those described above for layer (A), provided that the presence of said corresponding stimulatable copolymer or copolymers does not constitute an obstacle to rupture of the unilamellar membrane.

In exemplary embodiments, and relative to the total weight of copolymers in said membrane, the layer (B) includes less than 50% by weight, in particular less than 30% by weight or it may even be free of amphiphilic block copolymer that is sensitive to the exogenous stimulus that activates the layer (A) and more particularly to any exogenous stimulus.

Layer (B) may be primarily formed from copolymer(s) that are insensitive to an exogenous stimulus selected for layer (A).

Relative to the total weight of copolymers in said membrane, layer (B) may include in the range 10% to 95% by weight, preferably in the range 30% to 70% by weight of amphiphilic block copolymer(s) that is insensitive to an exogenous stimulus.

In particular advantageous exemplary embodiments of the vesicles of the invention:

layer (A) is formed from an amount of at least 70% by weight of stimulatable amphiphilic block copolymer relative to the total weight of copolymer(s) in the unilamellar membrane; and

layer (B) is formed from an amount of at least 70% by weight of at least one non-stimulatable copolymer relative to the total weight of copolymer(s) in the unilamellar membrane.

More particularly, layer (A) comprises, as the stimulatable amphiphilic block copolymer, the block copolymer poly(ethyleneglycol)-b-poly(4-butyloxy-2′-(4-(methacryloyloxy)butyloxy)-4′-(4-butyloxy-benzoyloxy)azobenzene (denoted PEG-b-PMAazo444), in which the PMAazo444 block is sensitive to UV light.

More particularly, layer (B) is essentially formed from poly(ethyleneglycol)-b-poly(butadiene) (termed PEG-b-PBD) that is insensitive to UV light.

In exemplary embodiments, layer (A) constitutes the outer layer of said vesicle and layer (B) constitutes the inner layer.

In other exemplary embodiments, layer (A) constitutes the inner layer of said vesicle and layer (B) constitutes the outer layer.

Advantageously, the vesicles of the invention have an architecture that favors the presence in the outer layer of amphiphilic block copolymers provided with biocompatible hydrophilic blocks, i.e. compatible with application, whether topical or in vivo, to the human or animal body. A preferred biocompatible hydrophilic block that may be mentioned is polyethylene glycol.

A vesicle of the invention including a “core” comprising a liquid phase, in particular an aqueous liquid phase, may also include in said liquid phase at least one active ingredient, in particular a hydrosoluble active ingredient. This may then be termed a vesicle with an encapsulated active ingredient or active ingredients in accordance with the invention.

### Active Ingredient

The active ingredient may be a hydrophilic active ingredient, that is, for example, selected from cosmetic active ingredients, dermatological active ingredients, pharmaceutical active ingredients, phytopathological active ingredients, polymers, and/or chemical reagents.

The active ingredients may, for example, be selected from polymers, pesticides, fungicides, chemical reagents such as oxidizing agents or reducing agents, ferrofluids, and mixtures thereof.

More particularly, in the pharmaceutical or cosmetic field, the active ingredient may be selected from moisturizing agents, desquamating agents, colorants, nutrients, sugars, salts, electrolytes, enzymes, vitamins, proteins or fragments of proteins, genes or fragments of genes, genetic engineering products, steroids, adjuvants, soothing agents and/or anti-irritants, astringent agents, healing agents, anti-inflammatories, anti-acne agents, antioxidants, dermo-relaxing agents, and antibacterials.

The quantity of active ingredient(s) in the vesicle of the invention is that conventionally used in the fields concerned.

The polymersomes of the invention may advantageously be used in the following fields of application: catalytic reactors (example: increasing yield in polymer synthesis); macromolecular industrial processes; depollution; photoreactors.

In accordance with other particular exemplary embodiments, the polymersomes of the invention are particularly advantageous in the following fields of application: environment (clean processes, automotive depollution, elimination of volatile organic compounds (VOC)); energy; fine chemicals; nanotechnologies; drug delivery; solar reactors.

Among the active ingredients in connection to the above-cited fields of application, mention can be made of quantum dots, electrolytes, polyelectrolytes, inorganic precursors solution for inorganic materials (such as TiCl₄, Si(OEt)₃, etc.), catalysts of controlled radical polymerizations, monomers (ex. monomers for conductive or semi-conductive polymers, etc.), organic reactants . . . .

The vesicles with an asymmetrical unilamellar membrane in accordance with the invention may be manufactured using any method that allows the production of distinct layers (A) and (B), said method consisting in particular in assembling each layer (A) and (B) independently of the other, in order to form an asymmetrical unilamellar vesicle.

### Production Method

As indicated above, the method of producing the vesicles with an asymmetrical unilamellar membrane in accordance with the invention implements the following:

dispersing an aqueous phase in an oily phase in the presence of at least one amphiphilic block copolymer (X) to form a layer of said amphiphilic block copolymer (X) around the aqueous droplets; and

subsequently, forming a layer comprising at least one amphiphilic block copolymer (Y) that is different from the amphiphilic block copolymer (X) around said coated droplets;

with only one of the two preceding steps using as the amphiphilic block copolymer an effective quantity of block copolymer that is capable of being stimulated by an exogenous stimulus.

More particularly, the polymersomes of the invention may be obtained using the following technique, which takes its inspiration from the method of synthesizing a lipid vesicle with an asymmetrical membrane described in the document “Engineering asymmetric vesicles”, PNAS, 16 Sep. 2003, vol. 100, N° 19, pages 10718-10721.

It is based on the use of a three-phase system involving the following phases:

### Firstly;

an upper liquid oily phase (O1), preferably toluene, comprising at least one amphiphilic block copolymer (Y), intended to constitute mainly the outer layer of a polymersome in accordance with the invention;

a lower aqueous phase (W1) that is contiguous with the upper liquid oily phase (O1), said lower aqueous phase (W1) being denser than the oily phase (O1); with, at the intersection of the upper oily phase (O1) and the lower aqueous phase (W1), an interphase layer formed by the amphiphilic block copolymer (Y) deriving from the upper oily layer (O1); and secondly

a third phase, formed by dispersing an aqueous phase (W2) in a liquid oily phase (O2), preferably toluene, in the presence of at least one amphiphilic block copolymer (X), which is distinct from the preceding amphiphilic block copolymer (Y) with only one of said copolymers (X) or (Y) being stimulatable by an exogenous stimulus. More precisely, said third phase contains aqueous droplets coated with a layer of amphiphilic block copolymers (X), essentially intended to form the inner layer of the polymersome of the invention.

The method involves bringing said third phase into contact with the upper face of the edifice formed by the superposed preceding two phases, namely the phase (O1) on the phase (W1); the vesicles of the invention are then obtained when said coated aqueous droplets present in the third phase pass through said interphase layer of (Y) copolymers. The molecules of (Y) copolymers organize themselves around these coated aqueous droplets to form the outer layer of the unilamellar membrane, the inner layer being formed by the (X) copolymers coating said aqueous droplets. The now doubly coated vesicles are recovered in the lower aqueous phase (W1).

Depending on the desired application, various active ingredients may be encapsulated in the vesicles of the invention.

In general, the active ingredients are hydrosoluble and are present in the aqueous phase (W2) of the initial reverse emulsion.

As discussed above, the vesicles of the invention containing at least one active ingredient or the compositions comprising said vesicles are of particular use for the purposes of encapsulation, transport, vectorization, and/or release of active ingredient(s), in particular hydrosoluble active ingredients such as pharmacological molecules, dermatological molecules, cosmetic molecules, synthetic or natural polymers, bactericides, pesticides, fungicides, and/or chemical reagents. This encapsulation, transport, vectorization, and/or release of active ingredients is preferably carried out in vivo, ex vivo, in particular on keratinous materials such as the skin or keratinous fibers, or in vitro, for controlled chemistry applications (reaction in a defined time and location) and in the microfluidics field.

### Controlled Release

The controlled release of active ingredient(s) encapsulated in one or more polymersome(s) in accordance with the invention consists in exposing said polymersome or polymersomes to an exogenous stimulus that is reactive having regard to at least one stimulatable copolymer forming one of the layers (A) and (B).

The term “exogenous stimulus” as used in the context of the invention means an external physical or chemical agent, preferably physical, capable of provoking a response in an excitable asymmetrical unilamellar membrane.

It should be understood that the nature of this stimulus is directly linked to the type of stimulatable copolymer(s) used in the vesicle of the invention.

The skilled person will take care to select the suitable stimulus. The stimulus will in fact be selected as a function of the nature of the stimulatable amphiphilic block copolymer, certain copolymers having a more pronounced sensitivity for certain stimuli than others, and/or of the type of application envisaged for the polymersomes of the invention.

The exogenous stimulus may, for example, be selected from UV light, infrared light, an electrical field, a magnetic field, and temperature, in particular a triggering temperature of between 20° C. and 100° C. and preferably between 30° C. and 80° C. that may be modulated by the chemical structure of the stimulatable copolymer.

Preferably, the exogenous stimulatable is UV light.

This exogenous stimulus must be exerted on the vesicles of the invention using an intensity and duration that is sufficient and dependent on the nature of the stimulatable amphiphilic block copolymers in order to cause said vesicles to burst by a modification to the steric configuration of the stimulatable copolymer induced, for example, by a change in the azobenzene motif from a trans configuration to a cis configuration. Without wishing to be bound by one theory, it appears that this modification to the configuration of an effective quantity of stimulatable copolymers generates an excess of surface area in just one of the two layers (A) and (B) and thus a change in the curvature that is sufficiently large within the unilamellar membrane of the vesicle to provoke the rupture thereof, the consequence being that said vesicle bursts and said encapsulated active ingredient or active ingredients is/are released.

It should be noted that this phenomenon occurs regardless of the location of the stimulatable copolymer, namely in the inner layer or in the outer layer.

This controlled release method may be carried out (i) in vitro, in a macroscopic, or microfluidic, or even nanofluidic environment, (ii) in a living organism (in vivo) in target organs, or (iii) on a living organism (ex vivo). In this implementation, the vesicles may, for example, be applied topically via a composition containing them.

The following examples and figures are given by way of non-limiting illustration of the invention. The abbreviations PEG-b-PMAazo444, PEG-b-PA444, and PEG-b-PBD respectively mean the block copolymer polyethylene glycol-b-poly((4-butyloxy-2′-(4-(methacryloyloxy)butyloxy)-4′-(4-butyloxy-benzoyloxy)azobenzene, the block copolymer polyethylene glycol-b-poly(4″-acryloxybutyl 2,5-di(4′-butyloxy-benzoyloxy)benzoate, and the block copolymer polyethyleneglycol-b-polybutadiene.

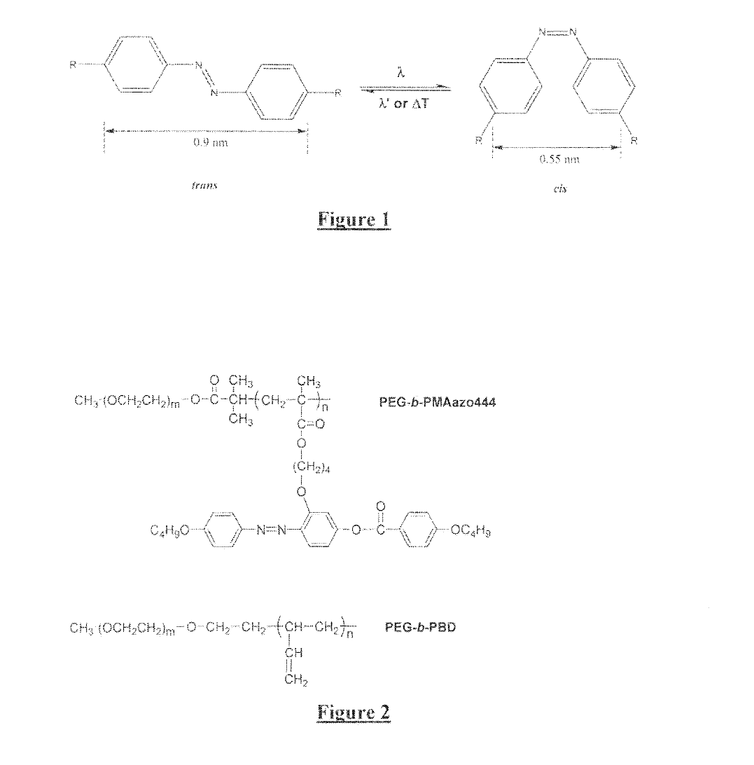

FIG. 1 represents the reversible isomerization of an azobenzene molecule.

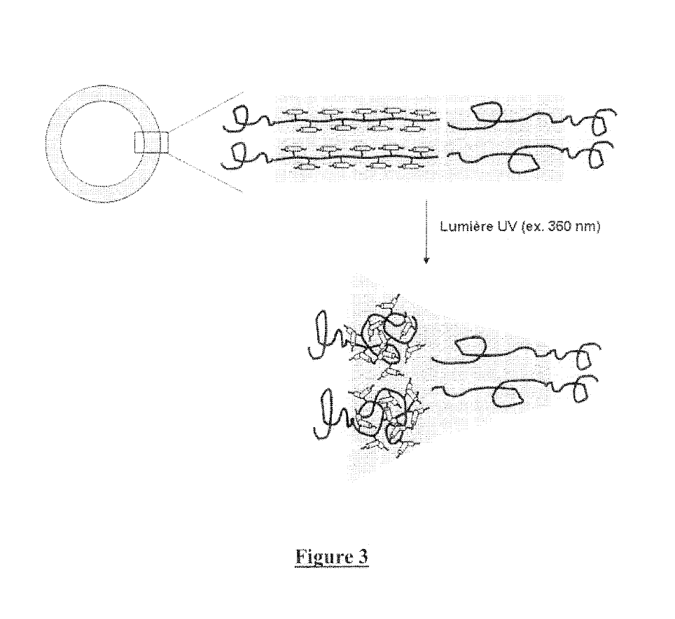

FIG. 2 represents the chemical structures PEG-b-PMAazo444 and PEG-b-PBD.

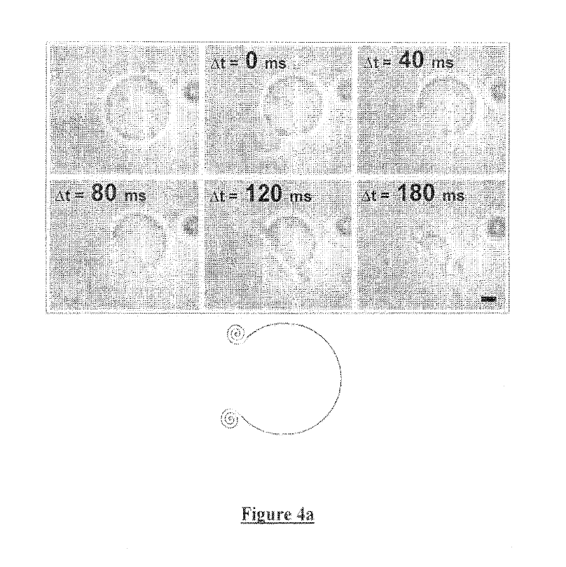

FIG. 3 is a schematic diagram of the change in shape of a photo-stimulatable liquid crystal block in layer (A) of the stimulatable asymmetrical polymersome membrane, the straight cylinders representing the motifs containing the azobenzene groups with a liquid crystal nature before UV irradiation and the bent cylinders representing the motifs containing the azobenzene groups after UV irradiation, having lost their liquid crystal character.

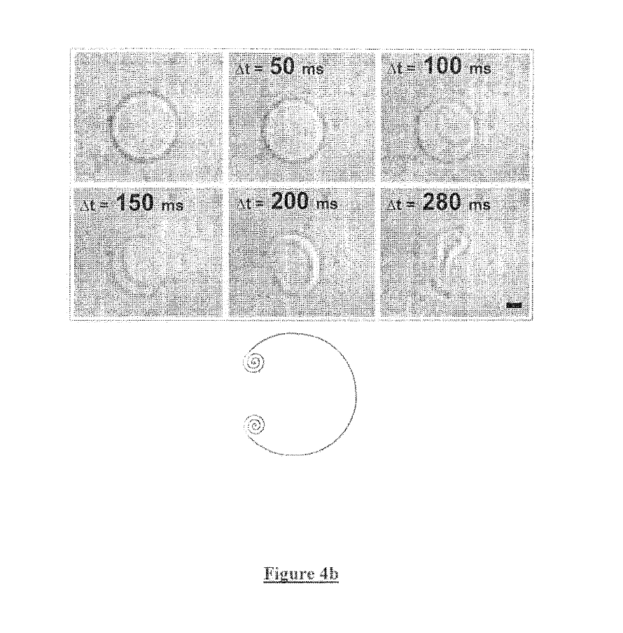

FIGS. 4a and 4b are photographs showing the bursting sequences of polymersomes as well as diagrams illustrating this bursting with stimulatable layer (A) as the inner layer for FIG. 4a or with layer (A) as the outer layer in FIG. 4b. The bar scale in FIGS. 4a and 4b represents 10 μm. The time Δt=0 corresponds to the moment when bursting of the polymersome commences and when the encapsulated sucrose is released.

### EXAMPLE 1

Method of Synthesizing a Vesicle with an Asymmetrical Unilamellar Membrane in Accordance with the Invention

In the following text, the copolymer (X) was a PEG-b-PMAazo444, the synthesis of which is described in Chem. Commun., 2005, 4345-4347, and Liq. Cryst., 2000, 27, 1497, and the compound (Y) was a PEG-b-PBD (sold by Polymer Source) (Mn=2300/5000).

The method of synthesizing the polymersomes of Examples 1 to 4 derives from an adaptation of the method described in Langmuir, 2003, 19, 2870.

More precisely, when preparing polymersomes in accordance with the invention, i.e. such as those used in Example 2, the following were carried out:

a) A first oily phase was prepared by sonication and dissolution of a copolymer (X) at a concentration of 3 mg/mL [milligram/millimeter] in toluene at 50° C. for 2 hours.

At the same time, an aqueous solution was also prepared from a buffer solution containing 380 mOsm [milliOsmolarity] of sucrose, supplemented with Dextran (commercially available from SIGMA, Mw=40000 g/mol [gram/mole] in a concentration of 100 mg per mL. 5 μL of this aqueous solution was then added to 500 μL of the preceding oily phase in a centrifugation tube (EPPENDORF); reverse emulsification was carried out under mild conditions by repeated pipetting using a syringe.

b) At the same time, 30 μL of a 380 mOsm solution of glucose was placed in a centrifugation tube and 30 μL of an oily phase containing the copolymer (Y), prepared by sonication and dissolving a copolymer (Y) at a concentration of 3 mg/mL in toluene at 50° C. for 2 hours, was gently added to the surface of this aqueous phase. Because of their amphiphilic nature, the (Y) copolymers tended to diffuse from the oily phase towards the interface. Complete coverage of the interface between the oily phase and the aqueous phase was accomplished after 5 minutes using copolymer (Y).

c) Finally, 50 μL of the emulsion prepared in a) was transferred to the surface of the centrifugation tube prepared in accordance with b) and the assembly was then immediately and instantaneously centrifuged (54 17R, EPPENDORF), selecting one of the following cycles: 100 g [grams] for 12 min, 350 g for 8 min then 500 g for 3 min, or 100 g for 12 min. Comparatively, the first cycle provided a better yield in terms of number of vesicles; the second cycle provided a population of vesicles with a slightly larger diameter. Centrifuging had the advantage of accelerating sedimentation of the droplets of the reverse emulsion across the interface that was covered by the monolayer of amphiphilic polymer and thus the formation of the expected polymersomes, namely polymersomes with an asymmetrical unilamellar membrane comprising an outer layer (A) of PEG-b-PMAazo444, an inner layer (B) of PEG-b-PBD and sucrose as the encapsulated active ingredient.

### EXAMPLE 2

### Demonstrating the Controlled Release Process for Sucrose

The following method was used to observe this release: a solution (aqueous external, glucose with identical osmolarity to the aqueous solution of sucrose composing the internal medium of the vesicles; osmolarity in the range 20 to 500 mOsm and preferably approximately 350 mOsm), comprising polymersomes with an asymmetrical unilamellar membrane comprising an outer layer (A) of PEG-b-PMAazo444, an inner layer (B) of PEG-b-PBD and sucrose as the encapsulated active ingredient, was prepared as described in Example 1.

At the same time, a solution including polymersomes comprising an inner layer (A) of PEG-b-PMAazo444, an outer layer (B) of PEG-b-PBD and sucrose as the encapsulated active ingredient was also prepared, said polymersomes being synthesized using a method similar to that described in Example 1 with PEG-b-PBD as the copolymer (X) and PEG-b-PMAazo444 as the copolymer (Y).

The solutions were each introduced into a sealed cell composed of two glass slides spaced apart by a spacer, for example Vitrex sealing paste. Next, the cells were illuminated through the objective of a microscope using a conventional UV lamp provided with a 360 nm filter (U-360 Band Filter, Edmund Optics), and optionally a heat-reflective mirror (Mirror Hot 0deg 50MM SQ, Edmund Optics) if heating of the cell was to be limited.

After illuminating for 30 seconds to 4 minutes, explosion/total defragmentation of the exposed polymersomes was observed with liberation of sucrose, knowing that the exposure time which preceded explosion of the vesicles was inversely proportional to the power of the lamp.

The optics of the microscope was preferably adapted to relay the maximum amount of UV light (UV objective—Olympus, 40×, UApo 340; UV reflective mirrors—Omega).

### COMPARATIVE EXAMPLES 3 AND 4

A solution (external aqueous glucose solution, osmolarity identical to the aqueous solution of sucrose composing the internal medium of the vesicles; osmolarity in the range 20 mOsm to 500 mOsm, preferably approximately 350 mOsm) was prepared, comprising polymersomes with a symmetrical unilamellar membrane comprising an outer layer and an inner layer of PEG-b-PMAazo444 and sucrose as the encapsulated active ingredient (comparative example 3) as well as a solution comprising polymersomes with a symmetrical unilamellar membrane comprising an outer layer and an inner layer of PEG-b-PBD and sucrose as an encapsulated active ingredient (comparative example 4).

The two types of polymersomes were synthesized using a method similar to that described above in Example 1 but using PEG-b-PMAazo444 as the copolymer (X) and copolymer (Y) for comparative Example 3 and using PEG-b-PBD as copolymer (X) and copolymer (Y) for comparative Example 4.

These solutions were each introduced into a sealed cell composed of two glass slides spaced apart by a spacer. Next, the cells were illuminated through the objective of a microscope using a conventional UV lamp provided with a 360 nm filter (U-360 Band Filter, Edmund Optics), and a heat-reflective mirror (Mirror Hot 0deg 50MM SQ, Edmund Optics).

Regarding the solution of symmetrical polymersomes based on PEG-b-PMAazo444 (comparative Example 3), a reduction in their volume was observed for a population of said vesicles in the range 30% to 70%, at the end of 1 minute to 4 minutes of illumination but without ever arriving at an ultimate explosion/defragmentation stage. Further, after having reached a certain minimum volume, the vesicles no longer changed despite maintaining the illumination for a further 10 minutes.

Regarding the solution of symmetrical polymersomes based on PEG-b-PBD (comparative Example 4), no modification in the polymersomes was observed after 15 minutes exposure to illumination with a conventional UV lamp provided with a 360 nm filter (U-360 Band Filter, Edmund Optics) and a heat reflecting mirror (Mirror Hot 0deg 50MM SQ, Edmund Optics) under conditions identical to those used in Example 2.

1. A vesicle with an asymmetrical unilamellar membrane, said membrane being constituted by two distinct layers, a layer (A) and a layer (B), said layers being superposed and each comprising at least one respective amphiphilic block copolymer, wherein said layer (A) comprises an effective quantity of amphiphilic block copolymer(s) comprising at least one block that is capable of being stimulated by an exogenous stimulus, said stimulatable blocks provided by said copolymers being capable of adopting, in response to said exogenous stimulus, a novel steric configuration conditioning rupture of said unilamellar membrane, and wherein said layer (B) does not have an effective quantity of amphiphilic block copolymer(s) comprising at least one block that is capable of being stimulated by said exogenous stimulus.

2. The vesicle according to claim 1, wherein the stimulatable amphiphilic block copolymer is selected from copolymers comprising at least one hydrophilic block and at least one photo-stimulatable liquid crystal hydrophobic block.

3. The vesicle according to claim 2, wherein the photo-stimulatable liquid crystal hydrophobic block comprises at least one azobenzene group.

4. The vesicle according to claim 3, wherein the photo-stimulatable liquid crystal hydrophobic block is selected from poly(4-butyloxy-2′-(4-(methacryloyloxy)butyloxy)-4′-(4-butyloxybenzoyloxy)azobenzene), poly(4-butyl-2′-(4-(methacryloyloxy)butyloxy)-4′-(4-butyloxy-benzoyloxy)azobenzene, poly(4-butyloxy-2′-(4-(acryloyloxy)butyloxy)-4′-(4-butyloxy-benzoyloxy)azobenzene, poly(4-butyl-2′-(4-(acryloyloxy)butyloxy)-4′-(4-butyloxy-benzoyloxy)azobenzene, poly(4-alkyloxy-2′-(methacryloyloxyalkyloxy)-4′-(4-alkyloxy-benzoyloxy)azobenzene, poly(4-alkyloxy-2′-(acryloyloxyalkyloxy)-4′-(4-alkyloxy-benzoyloxy)azobenzene, poly(4-alkyl-2′-(methacryloyloxyalkyloxy)-4′-(4-alkyloxy-benzoyloxy)azobenzene, poly(4-alkyl-2′-(acryloyloxyalkyloxy)-4′-(4-alkyloxy-benzoyloxy)azobenzene, poly(4-alkyl-2′-(methacryloyloxyalkyloxy)-4′-(4-alkyl-benzoyloxy)azobenzene, poly(4-alkyl-2′-(acryloyloxyalkyloxy)-4′-(4-alkyl-benzoyloxy)azobenzene, poly(4-alkyloxy-2′-(methacryloyloxyalkyloxy)-4′-(4-alkyl-benzoyloxy)azobenzene, poly(4-alkyloxy-2′-(acryloyloxyalkyloxy)-4′-(4-alkyl-benzoyloxy)azobenzene, poly(6-[4-(4-methoxyphenylazo)phenoxy]hexylmethacrylate, and poly(6-[4-(4-methoxyphenylazo)phenoxy]hexylacroylate.

5. The vesicle according to claim 2, wherein the hydrophobic block is selected from polyethylene glycol, polyacrylic acid, polymethacrylic acid, poly(N-alkylacrylamide), poly(N-alkylaminoacrylate), poly(acrylic acid-co-oligo(ethylene glycol) acrylate), poly(methacrylic acid-co-oligo(ethylene glycol)methacrylate), poly(oligo(ethylene glycol)methacrylate), poly(oligo(ethylene glycol)acrylate), and poly(2-(2′-methoxyethoxy)ethylmethacrylate-co-oligo(ethylene glycol)methacrylate).

6. The vesicle according to claim 1, wherein layer (B) is primarily formed from copolymer(s) that are insensitive to said exogenous stimulus.

7. The vesicle according to claim 1, wherein layer (B) is formed from at least one copolymer selected from polyethylene glycol-polybutadiene block copolymers, polyacrylic acid-polystyrene block copolymers, polyethylene glycol-polystyrene block copolymers, polyethylene glycol-polyethylethylene block copolymers, polyethylene glycol-polybutadiene block copolymers, polyethylene glycol-poly-L-lactic acid block copolymers, and polyethylene glycol-polycaprolactone block copolymers.

8. The vesicle according to claim 1, wherein:
layer (A) is formed from an amount of at least 70% by weight of stimulatable amphiphilic block copolymer relative to the total weight of copolymer(s) in the unilamellar membrane; and
layer (B) is formed from an amount of at least 70% by weight of at least one non-stimulatable copolymer relative to the total weight of copolymer(s) in the unilamellar membrane.

9. The vesicle according to claim 1, wherein:
layer (A) comprises, at least as the stimulatable amphiphilic block copolymer, poly(ethyleneglycol)-b-poly(4-butyloxy-2′-(4-(methacryloyloxy)butyloxy)-4′-(4-butyloxy-benzoyloxy)azobenzene, that is sensitive to UV light;
layer (B) is essentially formed from poly(ethyleneglycol)-b-poly(butadiene) that is insensitive to UV light.

10. The vesicle according to claim 1, wherein the exogenous stimulus is selected from UV light, infrared light, an electric field, a magnetic field, and temperature.

11. The vesicle according to claim 1, wherein, relative to the total weight of copolymer(s) in the asymmetrical unilamellar membrane, the layer (A) of the vesicle comprises more than 10% by weight.

12. The vesicle according to claim 1, wherein, relative to the total weight of copolymer(s) in the asymmetrical unilamellar membrane, the layer (B) of the vesicle includes in the range 10% to 95% by weight of amphiphilic block copolymer(s) insensitive to an exogenous stimulus.

13. The vesicle according to claim 1, wherein its mean diameter is in the range 100 μm to 20 nm.

14. The vesicle according to claim 1, wherein said vesicle further comprises at least one hydrophilic active ingredient.

15. A composition comprising at least one vesicle with an asymmetrical unilamellar membrane, said membrane being constituted by two distinct layers, a layer (A) and a layer (B), said layers being superposed and each comprising at least one respective amphiphilic block copolymer, wherein layer (A) comprises an effective quantity of amphiphilic block copolymer(s) comprising at least one block that is capable of being stimulated by an exogenous stimulus, said stimulatable blocks provided by said copolymers being capable of adopting, in response to said exogenous stimulus, a novel steric configuration conditioning rupture of said unilamellar membrane, and wherein said layer (B) does not have an effective quantity of amphiphilic block copolymer(s) comprising at least one block that is capable of being stimulated by said exogenous stimulus, in a hydrophilic or physiologically acceptable medium.

16. A method of producing vesicles with an asymmetrical unilamellar membrane, said membrane being constituted by two distinct layers, a layer (A) and a layer (B), said layers being superposed and each comprising at least one respective amphiphilic block copolymer, wherein layer (A) comprises an effective quantity of amphiphilic block copolymer(s) comprising at least one block that is capable of being stimulated by an exogenous stimulus, said stimulatable blocks provided by said copolymers being capable of adopting, in response to said exogenous stimulus, a novel steric configuration conditioning rupture of said unilamellar membrane, and wherein said layer (B) does not have an effective quantity of amphiphilic block copolymer(s) comprising at least one block that is capable of being stimulated by said exogenous stimulus wherein said method comprises:
a) dispersing an aqueous phase in an oily phase in the presence of at least one amphiphilic block copolymer (X) under conditions propitious to the formation of a layer of said amphiphilic block copolymer (X) around aqueous droplets;
b) bringing said coated droplets obtained at the end of the preceding step into contact with at least one amphiphilic block copolymer (Y), which is different from the amphiphilic block copolymer (X), under conditions propitious to the formation of a layer comprising said amphiphilic block copolymer (Y) superposed on the layer formed in step a); and
c) recovering said vesicles with an asymmetrical unilamellar membrane in an aqueous phase; wherein only one of steps a) and b) uses, as the amphiphilic block copolymer, an effective quantity of at least one amphiphilic block copolymer comprising at least one block that is capable of being stimulated by an exogenous stimulus.

17. The method according to claim 16, wherein the formation of the layer of step b) takes place at the interface of an oily phase and an aqueous phase.

18. The method according to claim 16, wherein the other step does not have any amphiphilic block copolymer comprising at least one block that is capable of being stimulated by said exogenous stimulus.

19. A method of encapsulating at least one hydrophilic active ingredient, in vesicles with an asymmetrical unilamellar membrane, said membrane being constituted by two distinct layers, a layer (A) and a layer (B), said layers being superposed and each comprising at least one respective amphiphilic block copolymer, wherein layer (A) comprises an effective quantity of amphiphilic block copolymer(s) comprising at least one block that is capable of being stimulated by an exogenous stimulus, said stimulatable blocks provided by said copolymers being capable of adopting, in response to said exogenous stimulus, a novel steric configuration conditioning rupture of said unilamellar membrane, and wherein said layer (B) does not have an effective quantity of amphiphilic block copolymer(s) comprising at least one block that is capable of being stimulated by said exogenous stimulus, comprising:
a) dispersing an aqueous phase comprising at least one active ingredient in an oily phase in the presence of at least one amphiphilic block copolymer (X) under conditions that are propitious to the formation of a layer of said amphiphilic block copolymer (X) around aqueous droplets containing said active ingredient or active ingredients;
b) bringing said coated droplets obtained from the preceding step into the presence of at least one amphiphilic block copolymer (Y), which is different from the amphiphilic block copolymer (X), under conditions propitious to the formation of a layer comprising said amphiphilic block copolymer (Y) superposed on the layer formed in step a); and
c) recovering said vesicles with an asymmetrical unilamellar membrane containing said active ingredient or active ingredients in an aqueous phase; wherein only one of steps a) or b) employs, as the amphiphilic block copolymer, an effective quantity of at least one amphiphilic block copolymer comprising at least one block that is capable of being stimulated by an exogenous stimulus.

20. A method of the controlled release of at least one active ingredient contained in a vesicle with an asymmetrical unilamellar membrane, said membrane being constituted by two distinct layers, a layer (A) and a layer (B), said layers being superposed and each comprising at least one respective amphiphilic block copolymer, wherein said layer (A) comprises an effective quantity of amphiphilic block copolymer(s) comprising at least one block that is capable of being stimulated by an exogenous stimulus, said stimulatable blocks provided by said copolymers being capable of adopting, in response to said exogenous stimulus, a novel steric configuration conditioning rupture of said unilamellar membrane, and wherein said layer (B) does not have an effective quantity of amphiphilic block copolymer(s) comprising at least one block that is capable of being stimulated by said exogenous stimulus, and wherein the mean diameter of the said vesicle is in the range 100 μm to 20 nm, the method comprising exposing said vesicle to an exogenous stimulus, capable of inducing a modification of the steric configuration of the amphiphilic block copolymers comprising at least one block that is capable of being stimulated by said exogenous stimulus, under conditions sufficient to provoke rupture of said unilamellar membrane of said vesicle.

21. The method according to claim 20, wherein the exogenous stimulus is selected from UV light, infrared light, an electric field, a magnetic field, and temperature, which may be modulated by the chemical structure of the stimulatable copolymer.

22. A method for encapsulating, transporting, vectorizing and/or releasing at least one active ingredient in vitro, using a vesicle with an asymmetrical unilamellar membrane, said membrane being constituted by two distinct layers, a layer (A) and a layer (B), said layers being superposed and each comprising at least one respective amphiphilic block copolymer, wherein said layer (A) comprises an effective quantity of amphiphilic block copolymer(s) comprising at least one block that is capable of being stimulated by an exogenous stimulus, said stimulatable blocks provided by said copolymers being capable of adopting, in response to said exogenous stimulus, a novel steric configuration conditioning rupture of said unilamellar membrane, and wherein said layer (B) does not have an effective quantity of amphiphilic block copolymer(s) comprising at least one block that is capable of being stimulated by said exogenous stimulus.

23. The vesicle according to claim 1, wherein, relative to the total weight of copolymer(s) in the asymmetrical unilamellar membrane, the layer (A) of the vesicle comprises in the range 20% to 95% by weight of stimulatable amphiphilic block copolymer(s).

24. The vesicle according to claim 1, wherein, relative to the total weight of copolymer(s) in the asymmetrical unilamellar membrane, the layer (A) of the vesicle comprises in the range 30% to 70% by weight of stimulatable amphiphilic block copolymer(s).

25. The vesicle according to claim 1, wherein, relative to the total weight of copolymer(s) in the asymmetrical unilamellar membrane, the layer (A) of the vesicle comprises more than 40% by weight of stimulatable amphiphilic block copolymer(s).

26. The vesicle according to claim 1, wherein, relative to the total weight of copolymer(s) in the asymmetrical unilamellar membrane, the layer (B) of the vesicle comprises in the range 30% to 70% by weight of amphiphilic block copolymer(s) insensitive to an exogenous stimulus.

27. The vesicle according to claim 1, wherein its mean diameter is in the range 75 μm to 50 nm.

28. The vesicle according to claim 1, wherein its mean diameter is in the range 50 μm to 100 nm.

29. The vesicle according to claim 20, wherein the hydrophilic active ingredient is selected from polymers, chemical reagents and pharmaceutical active ingredients and phytopathological, dermatological and/or cosmetic active ingredients.

30. The method according to claim 26, wherein the hydrophilic active ingredient is selected from a cosmetic, dermatological or pharmaceutical molecule, a polymer and/or a chemical reagent.

31. The method according to claim 28, wherein the exogenous stimulus is with a triggering temperature in the range 20° C. to 100° C.

32. The method according to claim 28, wherein the exogenous stimulus is with a triggering temperature in the range 30° C. to 80° C.

33. The composition according to claim 15, wherein said composition is a chemical, cosmetic, dermatological or pharmaceutical composition.
